(19) United States
(12) Patent Application Publication
(10) Pub. No.: US 2016/0324994 A1
(43) Pub. Date: Nov. 10, 2016

(54) ULTRASONIC THERMAL DISINFECTOR

(71) Applicant: MOCOM S.R.L., Imola (Bologna) (IT)

(72) Inventors: Angelo BELLOSI, Imola (Bologna) (IT); Daniele TOSI, Vidigulfo (Pavia) (IT); Chiara ALBERTAZZI, Sant'agata Sul Santerno (Ravenna) (IT)

(73) Assignee: MOCOM S.R.L., Imola (Bologna) (IT)

(21) Appl. No.: 15/111,197

(22) PCT Filed: Mar. 26, 2015
(86) PCT No.: PCT/IB2015/052226

§ 371 Date: Jul. 13, 2016

(30) Foreign Application Priority Data
Apr. 2, 2014 (IT) BO2014A000185

(51) Int. Cl. A61L 2/04; B08B 3/12; A61C 19/00; B08B 3/02; A61B 90/70; A61L 2/025; B08B 3/10

(52) U.S. Cl. CPC A61L 2/04; A61L 2/025; B08B 3/12; B08B 3/102; B08B 3/02; A61B 90/70; A61C 19/002; A61L 2202/17; A61L 2202/24

(57) ABSTRACT

Provided is a thermal disinfector including a cover, a basin, at least an inlet for water, an outlet to drain the water into the sewer, an outlet for recirculating water so that it can be reintroduced into the basin, at least an ultrasound generator, an electronic control unit, and a water heater positioned outside the basin.

The present invention relates to a thermal disinfector provided with ultrasonic cleansing for the disinfection of dental instruments, or more generally, of medical/surgical instruments.

Generally, the gold standard in the treatment of medical/dental instruments is the sterilization through water steam autoclave; the efficacy of the sterilizing cycle is however linked to the state of cleanliness of the instruments which are inserted into the autoclave. Therefore, pre-treating the instruments before wrapping and inserting them into the autoclave is a common practice.

In the art two different apparatuses to pre-treat instruments to be sterilized have been known for a long time:
a) Ultrasonic baths for immersing instruments
   These are basins in which the instruments are immersed, where the instruments undergo the action of ultrasounds, which enable the removal of residual solids, especially plaster, dental cements, composite materials, present on the instruments themselves. Specific detergents/disinfectants may be added to the bath water.
b) Thermal disinfectors
   These are apparatuses which have a configuration very similar to traditional domestic dishwashers, which cleanse with water performing a strong mechanical action, at a high temperature (around 90° C.). In this case, too, specific detergents may be used.

Ultrasonic baths effectively clean instruments only, while thermal disinfectors ensure also a high degree of disinfection.

Both these apparatuses make it possible to decrease the risk to human operators linked to handling cutting/sharp instruments, and to better standardize the kind of treatment instruments undergo.

The disadvantage linked to thermal disinfector use is the duration of the disinfecting cycle, which, like in domestic dishwashers, is rather long-lasting, around two hours. Moreover, without the ultrasound cleansing, the removal of residual cement from instruments is more difficult, and therefore the operator has to remove it manually.

An apparatus that appeared more recently on the market is described in Soltec's patents IT1404342 and IT1404343. This apparatus is provided with a basin where instruments are immersed and undergo ultrasonic cleansing, with an optional thermal disinfection while the instruments are immersed. This kind of apparatus requires the heating of a large quantity of water, and therefore a long-lasting cycle duration. The basin is provided with a heating coil for heating the water in which the coil is immersed.

Another similar apparatus is described in patent application CN 201 423 361 Y, which describes a basin extending in a mainly vertical direction with heating means arranged on the bottom of and substantially inside said basin.

The present invention aims to provide an apparatus and a method to cleanse and disinfect instruments, improving the duration and the efficiency of the process, and protecting human operators.

This purpose is achieved by an apparatus and a method having the features of the independent claims. Advantageous embodiments and refinements are specified in the claims dependent thereon.

The apparatus of the present invention also consists of a basin closed by a cover wherein consecutive steps of a cycle are automatically performed:
Pre-wash with cold water;
Ultrasonic cleansing with instruments immersed at a temperature of 45-60° C.;
Rinsing;
Thermal disinfection;
Drying.

With respect to the prior art, the advantage of the present apparatus as compared to ultrasonic baths and thermal disinfectors is the better efficiency of the process. With respect to the apparatus described in Soltec's patents and in patent application CN 201 423 361 Y there is a lower use of water and a more rapid cycle. This is obtained thanks to the special heating system, which does not require the use of heating coils within the basin, and to the fact that the thermal disinfection step is not performed with immersed instruments, but is performed through water jets which recirculate the water present in the basin.

Further advantages and properties of the present invention are disclosed in the following description, in which exemplary embodiments of the present invention are explained in detail based on the drawings:

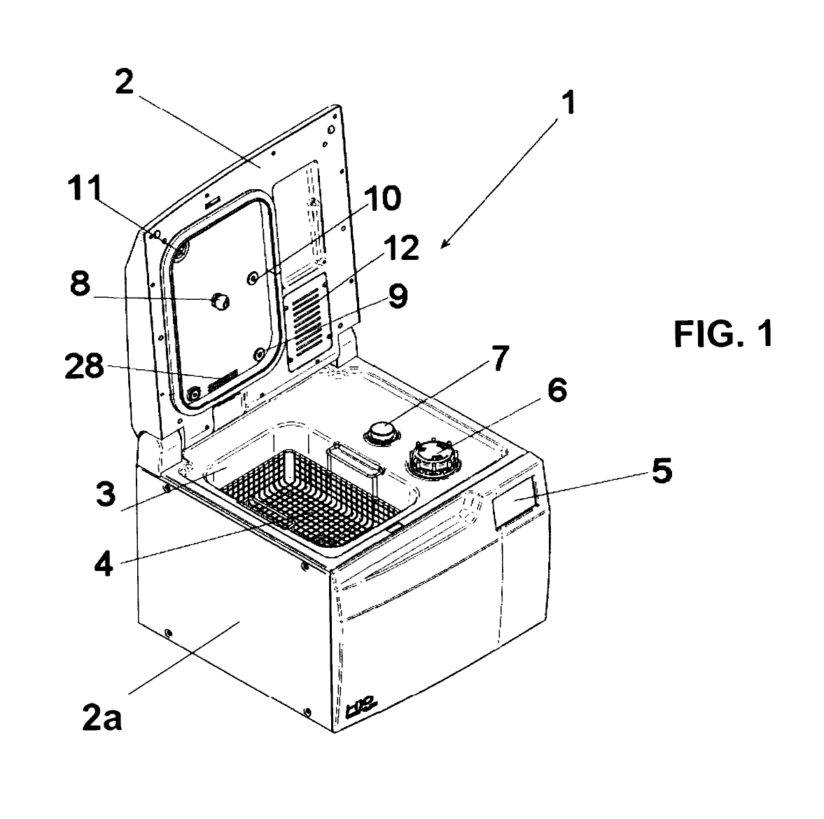

FIG. 1 Perspective view of the thermal disinfector;

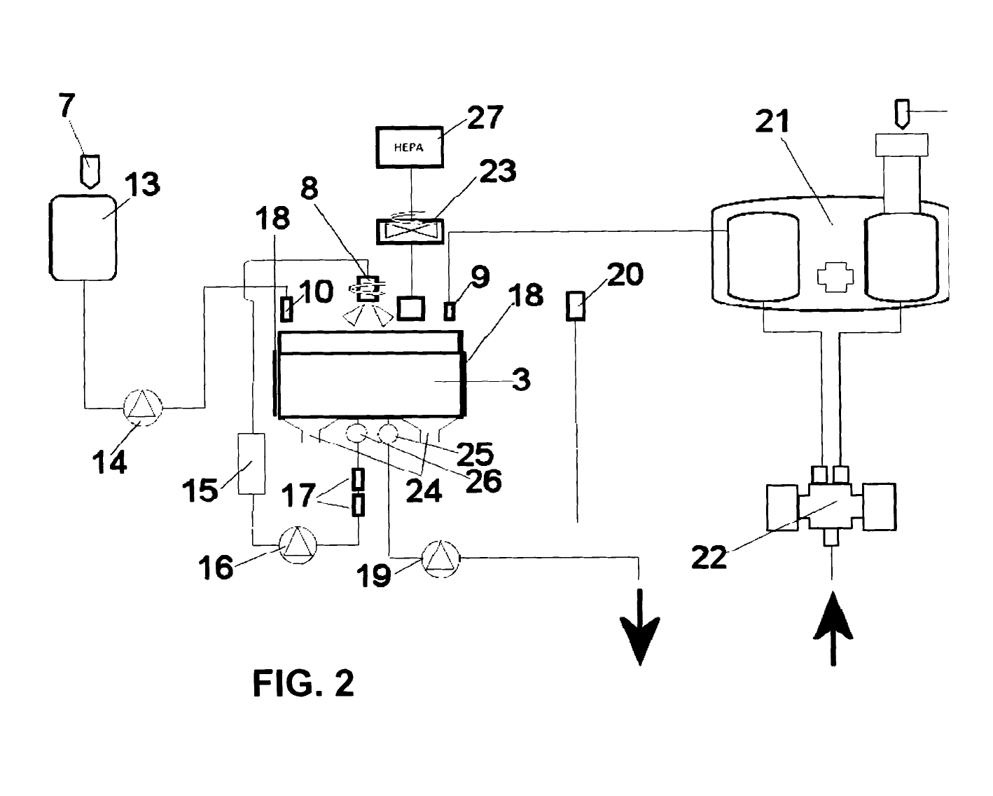

FIG. 2 Schematic diagram of the thermal disinfector;

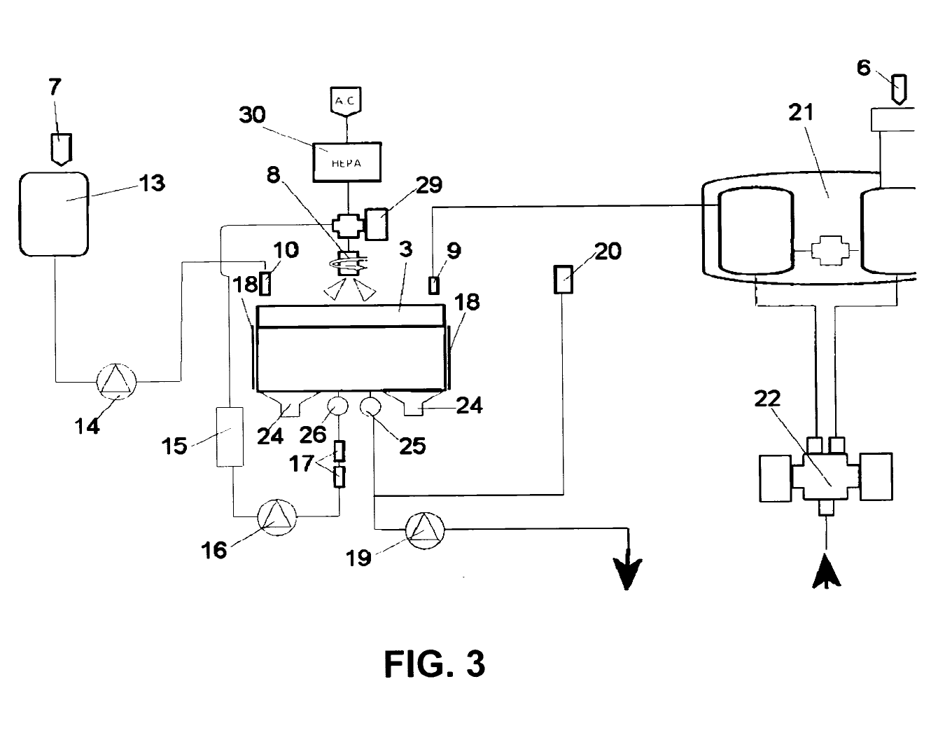

FIG. 3 Schematic diagram of a further embodiment of the thermal disinfector.

FIG. 1 shows a perspective view of the thermal disinfector of the present invention. The thermal disinfector 1 comprises a cover 2, a basin 3, a display 5.

Within the basin 3 there is a basket 4 where the instruments to be washed and disinfected are placed. On the surface beneath the cover 2 there is an inlet 6 to the reservoir of salt for the regeneration of the resins of a water softener, and an inlet 7 to the detergent reservoir.

Structurally the thermal disinfector 1 preferably comprises a container 2a, which preferably houses said basin 3, in a raised position with respect to the bottom of said container 2a, and the other elements. The container 2a is preferably entirely closed by the cover 2.

On the underside of the cover 2 there is at least a sprinkler 8 for inletting water jets into the basin, and also a nozzle 9 for inletting water coming from the water mains. Also on the underside of the cover 2 there is a port 11 for introducing air for the drying step, which flows out through the exit port 28. Under the grid 12 there is a HEPA filter to filter incoming air.

FIG. 2 shows a schematic diagram of the components of the thermal disinfector 1. The basin 3 is provided with a plurality of sonotrodes 24 for generating ultrasound. Moreover, the basin 3 is provided with external heating bands 18, consisting of electric resistors, for heating the walls of the basin 3.

The basin 3 extends preferably mainly in a horizontal plane, in particular the dimensions in the horizontal plane are more than three times greater than the dimensions in the vertical direction. The sonotrodes 24 are also preferably arranged in correspondence with the largest surface, that is to say in correspondence with the lower surface that extends in the horizontal plane.

The basin 3 can be filled to two different water levels:
A high level for immersing instruments during the ultrasound step;
A low level for the initial prewash step, rinsing step and thermal disinfection step.

Water can enter the basin 3 through two different inlets:
The nozzle 9, introducing water coming from the water mains (indicated with the upward bold arrow) into the basin 3 through a valve 22; said water is softened thanks to the passage through a softener group 21, and enters the basin at the room temperature of the mains water;
The sprinkler(s) 8, introducing recirculating water only into the basin 3, passing through a recirculating pump 16 and a water heater 15, which can be on or off. When the water heater 15 is off, it does not heat the water.

Incoming water, in any quantity, is always initially introduced through the nozzle 9. The basin 3 is provided with two outlets for water: an outlet 26 which is the outlet for recirculating water, and an outlet 25, which connects the basin to the drain and eventually to the sewer; said final outlet is indicated with the downward bold arrow. On the drainage line from the outlet 25 a connection branches off, at whose end is positioned a level sensor 20 which detects the water level in the basin 3. Also on the drainage line there is a pump 19 for extracting water from the basin 3.

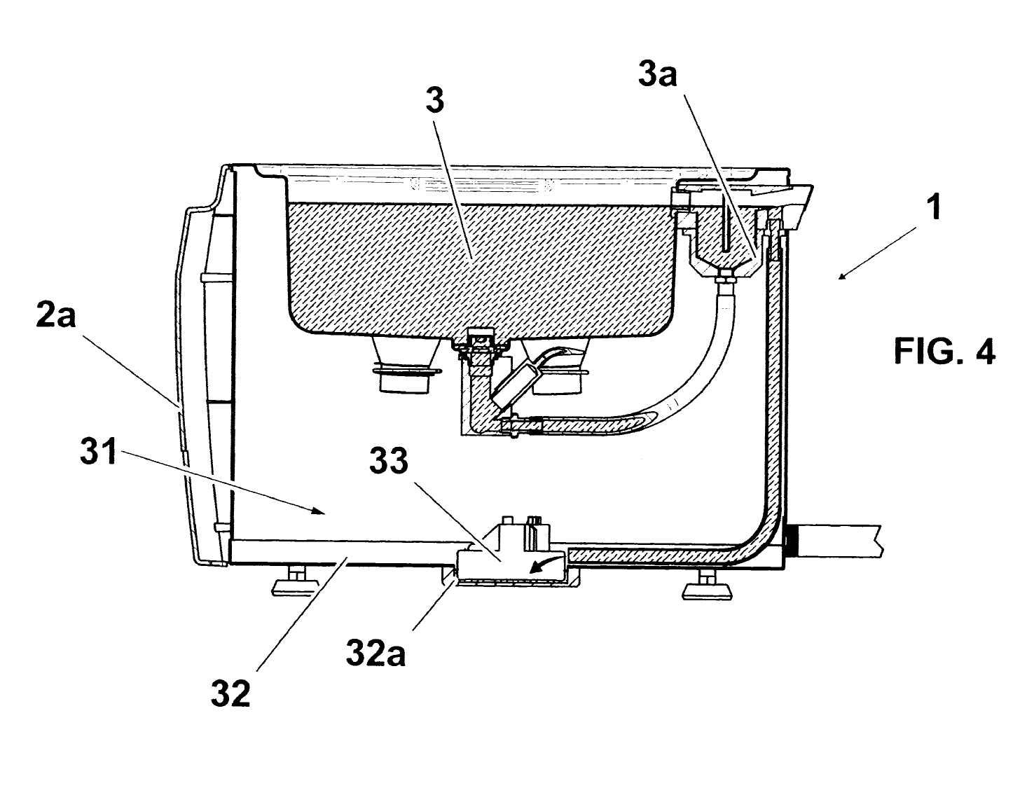

The thermal disinfector 1 also, preferably, comprises anti-flooding means 31 (FIG. 4), preferably comprising a containment tray 32 arranged on the bottom of the container 2a and suitable to contain the water that flows out through broken pipes or from the basin 3 through an overflow hole 3a. The anti-flooding means 31 preferably comprise a float valve 33 arranged in said containment tray 32 and more preferably in a lowered portion 32a of the tray 32. The float valve 33 is preferably mechanically linked to a water shut-off valve, preferably a solenoid valve. Said solenoid valve is appropriately suitable to move and disconnect the water circuit, and the flow of water into the thermal disinfector 1 and the container 2a when the float valve 33 rises due to flooding.

Downstream of the recirculating water outlet 26 there is at least a temperature sensor 17 for detecting the temperature of the recirculating water.

In the thermal disinfector 1 there is a reservoir 13 for a detergent, which through a dosing pump 14 is directly introduced into the basin 3 through a specific inlet 10. Above the basin 3 there is a fan 23 supplied with air passing through a HEPA filter 27 intended to purify the air entering the basin; said air is used to dry the instruments, and is filtered so that it does not contaminate them in the final step of the cycle successive to the disinfection step. The incoming air also preferably passes through heating means, preferably comprising a labyrinth with heating fins, suitable to heat the air in order to dry the instruments quickly. The air inlet duct preferably also comprises a temperature sensor and a one-way valve that allows the air to enter the basin 3 but not to flow back out of it.

The position of the heater 15 outside and at a distance from the basin 3, and so not directly in contact with the basin 3, avoids the need for heating coils inside the basin, as is the case in the apparatus described in the aforesaid patents. This is advantageous in that it leaves the basin totally free, so that cleansing is easier and the safety of the thermal disinfector 1 is enhanced. This is an advantage also in terms of structural simplicity and reliability, in that the basin 3 does not exhibit holes for the passage of the ends of the resistors, which would require a watertight sealing, difficult to ensure given the presence of the vibrations caused by the sonotrodes 24.

Moreover, the recirculating water is heated as it passes through the water heater 15, and falls in the form of jets through sprinklers 8 on the instruments contained in the basket 4. This allows the use of smaller quantities of water, which are therefore heated more rapidly, significantly reducing the duration of the washing and thermal disinfection cycle.

The sprinklers 8 in one embodiment are present in the form of rotors, like those of a traditional domestic dishwasher; in the preferred embodiment, they are in the form of rotating nozzles. This is different from the fixed nozzles described in the aforesaid patent documents, in that their presence adds a mechanical action that significantly improves the washing and the removal of particles from instruments. Moreover, the sprinklers 8 sprinkle hot water in the basin 3 generating a whirl, thus improving the distribution of heat and detergent. Rotating nozzles generate a better water distribution as compared to rotors, using less water.

In practice, a human operator gathers the dirty instruments, suitably positions them in the basket 4, positions the filled basket 4 in the basin 3 and then closes the cover 2. Then she/he starts the automatic cycle using the display 5. The successive steps of the thermal disinfection cycle are automatically controlled by an electronic control unit (not shown).

The method applied by the ultrasonic thermal disinfector of the present invention comprises the following steps:
a) Introduction of water coming from the mains through the nozzle 9 via the softener group 21;
b) Cold cleansing through the sprinklers 8;
c) Release of dirty water through the outlet 25 towards the sewer;
d) Introduction of water coming from the mains through the nozzle 9 via the softener group 21; water is introduced up to its higher level;
e) Introduction of detergent, coming from the reservoir 13;
f) Heating of the liquid (water and detergent) present in the basin 3; in the preferred embodiment said heating occurs through the combined action of the heating bands 18 and of the outside water heater 15; at the same time ultrasonic washing is started; ultrasounds are generated by sonotrodes 24; thanks to recirculation, the water is progressively brought to about 45° C.; this step of ultrasonic washing requires at least 10 minutes;
g) Release of dirty water through the outlet 25 towards the sewer;
h) Introduction of water coming from the mains through the nozzle 9 via the softener group 21; water is introduced to its lower level;
i) Rinsing with clean water through the sprinklers 8 with recirculation of the water;
j) Release of dirty water through the outlet 25 towards the sewer;
k) Introduction of water coming from the mains through the nozzle 9 via the softener group 21; water is introduced up to its lower level;
l) Heating of the water through the combined action of the heating bands and the outside water heater 15;
m) The water is kept at the pre-set temperature for the contact time necessary to obtain thermal disinfection; in the preferred embodiment, at 90° C. for at least 5 minutes;
n) Release of dirty water through the outlet 25 towards the sewer;
o) Drying through air entering the basin 3 via the air port 11 passing through the HEPA filter 27; the air is released towards the environment through the air port 28.

In particular, concerning the ultrasonic cleansing step f), in the preferred embodiment water heating is preferably performed using the heating bands 18 and using water recirculation through the outside water heater 15 only for the minimum needed to mix the water and eliminate thermal stratification.

Concerning the drying step o), in a preferred embodiment shown in FIG. 3, the air is taken from outside the thermal disinfector 1, passes through a special HEPA filter 30 for compressed air, and is introduced into the basin 3 through the rotors or rotating nozzles 8. This makes it possible to improve air distribution, and more generally the drying step. Obviously the presence of a valve 29 is necessary, to allow the filtered air pipe to be connected to the last portion of the water pipe upstream of the sprinklers 8.

The total time of a cycle performed as described above is around 40 minutes. This is a very short time, if compared to the duration of a cycle of a traditional thermal disinfector, which requires a variable time of between 120-75 minutes. The above-described cycle makes it possible to obtain instruments free of residual solids, thanks to the mechanical action performed by the rotating nozzles and ultrasonic cleansing; moreover the instruments have a contamination level much lower than that obtainable with a simple ultrasonic bath without thermal disinfection.

Finally, the reduced quantity of water used thanks to recirculation allows heating to be performed very rapidly, and this significantly shortens the duration of the thermal disinfection cycle.
1. thermal disinfector
2. cover—2a container
3. basin—3a overflow hole
4. basket
5. display
6. salt reservoir inlet
7. detergent reservoir inlet
8. sprinklers
9. water inlet
10. detergent inlet port
11. air inlet port
12. HEPA filter grid
13. detergent reservoir
14. detergent dosing pump
15. heater
16. water recirculating pump
17. temperature sensors
18. heating bands
19. releasing pump
20. water pressure sensor
21. softener group
22. mains water inlet valve
23. drying fan
24. sonotrodes
25. water outlet
26. recirculating water outlet
27. HEPA filter
28. air outlet port
29. exchange valve
30. HEPA filter for compressed air
31. 31 anti-flooding means
32. tray—32a lowered portion
33. float valve

1.-14. (canceled)

15. A thermal disinfector, comprising:
a cover,
a basin,
at least an inlet for the water,
an outlet to drain the water to the sewer,
an outlet for water to be recirculated and reintroduced into the basin,
at least one ultrasound generator,
an electronic control unit, and
a water heater positioned outside of, and at a distance from, the basin.

16. The thermal disinfector according to claim 15, wherein the water is introduced into the basin following one of two distinct paths:
water coming from the mains passes through a softener group and enters the basin through the nozzle; or
water contained in the basin flows out through the recirculation outlet, passes through the outside water heater, enters the basin through at least one sprinkler; in said path the external water heater may be on, heating the water, or off, not heating it.

17. The thermal disinfector according to claim 15, wherein at least one of said water inlets transmits a mechanical action to the water.

18. The thermal disinfector according to claim 15, further comprising at least one heating band adhering to the basin.

19. The thermal disinfector according to claim 15, further comprising a HEPA filter to filter air coming from the environment, a port to introduce air into the basin, and a port for releasing air toward the environment.

20. The thermal disinfector according to claim 19, comprising means for heating said air introduced from the environment.

21. The thermal disinfector according to claim 18, further comprising an exchange valve which allows the air for drying the instruments to be conveyed through the rotating nozzles or rotors.

22. The thermal disinfector according to claim 15, wherein said basin extends mainly in the horizontal plane.

23. The thermal disinfector according to claim 22, wherein said sonotrodes are arranged in correspondence with the largest surface of said basin such that said sonotordes are correspondence with the lower surface which extends in said horizontal plane.

24. The thermal disinfector according to claim 15, comprising a container housing at least said basin in a raised position, further comprising anti-flooding means, comprising a containment tray arranged on the bottom of said container and comprising a float valve connected to a water shut-off valve, configured for preventing the water from entering the thermal disinfector when the float valve rises due to flooding.

25. A method for ultrasonic cleansing and thermal disinfection of instruments placed in the basket, contained inside the basin of the thermal disinfector comprising the following steps:
a) pre-wash with cold water;
b) cleansing of immersed or non-immersed instruments with or without ultrasounds, at a temperature of 45-60° C.;
c) rinsing;
d) thermal disinfection; and
e) drying
wherein the water is heated for step b) and step d) by recirculating it through the water heater arranged outside and at a distance from the basin.

26. The method for ultrasonic cleansing and thermal disinfection in said ultrasonic thermal disinfector according to claim 25, wherein the thermal disinfection step d) is performed keeping the water at a temperature of 80-93° C. for at least 5 minutes of contact time.

27. The method for ultrasonic cleansing and thermal disinfection in the ultrasonic thermal disinfector according to claim 25, wherein the water heating during said ultrasonic cleansing step b) is mainly performed using the heating bands and using water recirculation through the outside water heater for the minimum necessary to mix the water and eliminate thermal stratification.

28. The method for ultrasonic cleansing and thermal disinfection in the ultrasonic thermal disinfector according to claim 25, wherein the drying step e) is performed by introducing air into the basin (3) through rotors or rotating nozzles (8).

29. The thermal disinfector according to claim 17, wherein said water inlet is in the form of a rotating nozzle or rotor.
